(19) United States
(12) Patent Application Publication
(10) Pub. No.: US 2021/0369799 A1
(43) Pub. Date: Dec. 2, 2021

(54) NASAL COMPOSITIONS AND METHODS

(71) Applicant: YISSUM RESEARCH DEVELOPMENT COMPANY OF THE HEBREW UNIVERSITY OF JERUSALEM LTD., Jerusalem (IL)

(72) Inventors: Elka TOUITOU, Hod Hasharon (IL); Hiba NATSHEH, Jerusalem (IL)

(21) Appl. No.: 17/290,908

(22) PCT Filed: Oct. 31, 2019
(86) PCT No.: PCT/IL2019/051178

§ 371 Date: May 3, 2021

Related U.S. Application Data
(60) Provisional application No. 62/755,479, filed on Nov. 4, 2018.

(51) Int. Cl. A61K 36/185; A61K 9/00; A61K 45/06; A61K 47/24; A61K 47/10; A61P 25/28

(52) U.S. Cl. CPC A61K 36/185; A61K 9/0043; A61P 25/28; A61K 47/24; A61K 47/10; A61K 45/06

(57) ABSTRACT

A nasal composition comprising one or more vegetable oils that possess medicinal properties for the treatment or improvement of brain/central nervous system conditions and diseases; phospholipids and optionally glycol, the composition being cannabinoids-free.

The invention relates to nasally administrable compositions of vegetable oils possessing useful medicinal properties, including for central nerve system (CNS) and brain diseases therapy.

Certain vegetable oils possess useful therapeutic properties in their own right and may demonstrate a desired effect achieved through topical, systemic or CNS administration, for example, for slowing down brain degenerative processes and manage anxiety and depression. Such oils include black cumin seed oil, hemp seed oil, pomegranate seed oil, sesame seed oil, brassica seed oil and black sesame oil, to name a few. For example, pomegranate seed oil was shown to display neuroprotective effect (see Yuan et al. ACS Chem. Neurosci., 2016, 7 (1), pp 26-33). Recently pomegranate oil-containing food supplement (GranaGard®) was placed on the market; it is based sub-micron emulsion formulation that is able to cross the blood-brain barrier to transport the active components to the brain.

However, little has been reported on intranasal delivery of vegetable extracts, let alone therapeutically active seed oils for the purpose of achieving systemic effect or targeting the brain. Vegetable oils such as soybean oil, peanut oils, coconut oil, maize oil, olive oil and sunflower oil were used in intranasal aqueous compositions in combination with phospholipids for systemic delivery of polypeptides such as insulin and glucagon (see EP 272097). US 2010/0247694 and WO 2013/132487 both relate to a composition for topical application to the nasal membrane for relief from symptoms of sinusitis and rhinitis, mentioning pomegranate extract as active ingredient.

It has also been reported in US 2012/0156272 that vegetable extracts (including pomegranate extract) and phospholipids can be formulated into orally-administrable forms such as powders and granules and topical preparations such as creams and lotions. A complex consisting of standardized pomegranate extract and phospholipids was isolated in a solid form and characterized [Vora et al. Journal of Advanced Pharmaceutical Technology & Research 6(2), p. 75-80 (2015)].

As mentioned above, therapeutically useful vegetable oils could offer a set of benefits, for example, when reaching the brain, e.g., prevention or slowing down the development of neurological disorders at people at risk, e.g., elderly population.

We have now found compositions that are suitable for intranasal administration, enabling therapeutically beneficial oils to reach the brain/central nerve system (via the nasal route) and improve CNS aliments.

Accordingly, one aspect of the invention is a composition adapted for nasal administration comprising (i) one or more vegetable oils—especially seed oils—possessing medicinal properties (or isolated active fractions of such oils) and (ii) phospholipids. Optionally glycol may be added.

For example, oil(s): phospholipids weight ratio may vary from 1:0.25-1.5, e.g., from 1:0.25-1.25 and when glycol is present, oil(s): phospholipids:glycol weight ratio is in the range of 1:0.6-1.2:0.3-0.6.

It should be noted that the composition of the invention need not necessarily be used to deliver the therapeutically beneficial vegetable oils as sole active component; other active ingredients may be added to the composition. However, specific variant of the invention relates to a composition which is devoid of active ingredients other than the aforementioned vegetable oils, in particular, cannabinoids free compositions. That is, cannabinoids are not present in the composition of the invention (other than perhaps traces of cannabinoids which are sometimes found in commercial hemp seed oil—less 100 ppm THC and 10,000 ppm CBD, e.g., less than 10 ppm THC and 1000 ppm CBD).

In general, the concentration of the vegetable oils in the composition of the invention is from 0.5 to 75%, e.g., 1 to 75%. Exemplary ranges include from 0.5 to 5%, 0.5-30%, 5 to 15%, 5 to 75%, e.g., from 15 to 75%, 15 to 50%, based on the total weight of the composition. Vegetable oils are usually produced from plant seeds by methods such as pressing and solvent extraction. The oil can be rafinated or brut.

One preferred vegetable oil for use in the invention is pomegranate (Punica granatum) seed oil (PSO) that is produced by means of cold pressing, where the seeds are crushed and pressed to force the oil out; and solvent extraction, where the seeds are steam-heated, ground and repeatedly soaked with a suitable solvent such as hexane; see also U.S. Pat. No. 7,943,185 for suitable techniques. The major constituent of pomegranate seed oil is punicic acid, a polyunsaturated fatty acid [C18:3 9cis, 11trans, 13cis; see Journal of Human Nutrition & Food Science 2(1):1024, (2014), reporting the fatty acid composition of pomegranate seed oil obtained from several varieties].

Another preferred vegetable oil for use in the invention is hemp seed oil (HSO). It is produced by cold pressing the seeds of the Cannabis sativa and should not be confused with extractable materials made from the cannabis flower and leaves. Hemp seed oil may be used in the present invention either in a crude form (protein-containing) or in a refined form, following removal of the proteins. The composition of hemp seed oil is characterized by high content of polyunsaturated fatty acids:

TABLE A

| Major Fatty acids in HSO | % w/w | Saturation |
|---|---|---|
| Linoleic acid omega 6 | 50-70 | Polyunsaturated |
| α-Linoleic acid omega 3 | 15-25 | Polyunsaturated |
| γ-linoleic acid omega 6 | 1-6 | Polyunsaturated |
| Oleic acid | 10-16 | Monounsaturated |
| Palmitic acid | 5-9 | Saturated |
| Stearic acid | 2-3 | Saturated |

(based on Leizer et al. Journal of Nutraceuticals, Functional & Medical Foods Vol. 2(4) 2000 and U.S. Pat. No. 6,063,369). Good results are also obtained with sesame seed oil, olive oil and cumin seed oil. Mixtures consisting of two or more vegetable oils, such as pomegranate seed oil and olive oil; pomegranate seed oil and sesame seed oil; or pomegranate seed oil and hemp seed oil, are also contemplated by the present invention and corresponding formulations are illustrated below.

Turning now to the phospholipids, they are present in the composition of the invention at a concentration in the range from 15 to 80%, 15-70% by weight based on the total weight of the composition, for example, from 30 to 70%. Phospholipids suitable for use in the preparation of the composition according to the present invention include phosphoglycerides, e.g., phosphatidylcholine (PC). Other phospholipids can be selected from hydrogenated phosphatidylcholine, phosphatidylserine (PS), phosphatidylethanolamine (PE), phosphatidylglycerol, phosphatidylinositol and mixtures thereof. Lecithin, such as soya and egg lecithin, contains phosphatidylcholine. Suitable phosphatidylcholine-containing products produced with varying phosphatidylcholine content are commercially available, for example, from Lipoid under the brand names of Phospholipon® and Lipoid®, e.g., Lipoid S 20 (approx. 20-24% PC, 16-22% PE), Lipoid S45 (45% PC, 10-18% PE), Lipoid S75 (70% PC, 7-10% PE), Lipoid S80 (75% PC, 7% PE), Lipoid S100 (>94% PC), Phospholipon 50 (approx. 45-50% PC), Phospholipon 85G (>80% PC), Phospholipon 90G (>95% PC) or Phospholipids of rape, such as e. g. Lipoid R20 (approx. 20-24% PC, 16-22% PE), Lipoid R75 (70% PC, 7-10% PE), Lipoid R45 (45% PC, 10-18% PE), Lipoid R75 (75% PC, 7% PE), Lipoid R80 (75% PC, 7% PE), Lipoid R100 (>94% PC) or soy phospholipids from genetically non modified (non GMO) soy beans such as e. g. Lipoid P45 (45% PC, 10-18% PE), Lipoid P75 (75% PC, 7% PE), Lipoid P100 (>94% PC); sunflower phospholipids are also useful, e.g., from Perimondo under the brand names of Sunlipon®: Sunlipon® 90, Sunlipon® 65, Sunlipon® 50.

In the compositions of the invention, the phospholipid is combined with the oil, optionally together with a glycol such as propylene glycol. The constitutes may be combined with one another in any order, usually with gradual addition of the oil under stirring, to give a liquid, viscous liquid or gel-like preparation proportioned as described above. For example, the content of the phospholipid in said liquid, viscous liquid or gel-like preparation may be at least 30%, preferably at least 35%, e.g., from 40 to 70%, for example, from 40 to 60%. The oil(s) content may be at least 0.5%, e.g., at least 2%, 5%, 10%, and generally from 30 to 70% by weight based on the total weight of the composition. Such phospholipids/oils and phospholipids/oils/glycol admixtures may be used such are or incorporated into various nasal creams, nasal ointments, nasal suspensions and nasal gels in addition of course to nasal liquids, which may be used in suitable dispensing devices adapted for intranasal administration as describe below.

The mixing can be carried out by mixers, mortar and pestle or mortars such as Mortar Grinder RM200 (Retsch GmbH, Germany).

Antioxidants and auxiliary constituents can be added to the composition to serve various functions such as stability or increasing adherence to the nasal mucosa.

For example, one or more antioxidants are preferably added to the composition, e.g., at a concentration from 0.05 to 1.5% by weight based on the total weight of the composition. Suitable antioxidants include tocopherols and tocopheryl derivatives (vitamin E), 3,5-Di-tert-4-butylhydroxytoluene (BHT), butylated hydroxyanizole (BHA), vitamin C, sodium metabisulfite, potassium metabisulfite, ascorbic acid, lycopene, ascorbyl palmitate and the like. Mixtures of antioxidants may be used.

In addition to the components already listed above, the composition may further include auxiliary agents, such as surfactants, preservatives, thickening agents, viscosity and absorption enhancing agents, tolerance enhancers to reduce or prevent drying of the mucus membrane and to prevent irritation thereof.

Suitable preservatives that can be used with the composition include preservatives acceptable for nasal use, for example, benzyl benzalkonium salts.

Regarding thickening agents, the viscosity of the composition can be adjusted at a desired level using a pharmaceutically acceptable thickening agent.

Some illustrative compositions are set out below):
(i) from 40 to 70% (e.g., from 50 to 70%) by weight of one or more vegetable seed oil(s) such as pomegranate seed oil, sesame seed oil, olive oil, hemp seed oil and mixtures thereof, preferably pomegranate seed oil or a mixture thereof with a second oil; from 30 to 60% (e.g., from 30 to 40%) of phospholipids; and optionally from 0.1 to 1.5 antioxidant such as vitamin E.
(ii) from 1 to 20% (e.g., 5 to 20%) by weight of one or more vegetable seed oil(s) such as pomegranate seed oil and cumin seed oil; from 15 to 70% (e.g., 50 to 70%) of phospholipids; from 20 to 70% (e.g., 20 to 40%) of propylene glycol and optionally from 0.1 to 1.5 antioxidant such as vitamin E.

As used herein, nasally administering or nasal administration includes administering the compositions into nostrils of the nose to the mucous membranes of the nasal passage or nasal cavity of the mammal. For example, the compositions of the invention can be delivered to the nasal cavity as drops; liquid delivered to the nasal cavity as non-aerosol spray (packaged in a bottle with an atomizer attachment, such as a pump-sprayer) or as an aerosol spray packed in a container under pressure to emit pressurized suspension, as described in detail in Remington's Pharmaceutical Sciences (16th edition, Chapters 83 and 92). Suitable devices [nasal sprays, metered-dose sprays, squeeze bottles, liquid droppers, disposable one-dose droppers, nebulizers, cartridge systems with unit-dose ampoules, single dose pumps, bi-dose pumps, multiple-dose pumps] are of course commercially available from various sources. Regarding spray devices, it should be noted that both single (unit) dose or multiple dose systems may be used. Typically, a spray device comprises a bottle and a pump. The volume of liquid that is dispensed in a single spray actuation is in the range of from 5 to 250 microliters/each nostril/single administration and the concentration of the active ingredient in the formulation may be readily adjusted such that one or more spray into the nostrils will comply with the dosage regimen. Administration of compositions of the present invention may also take place using a nasal tampon or nasal sponge containing the compositions.

In the experimental work reported below, two techniques—multiphoton microscopy and near infrared (NIR) imaging—were used to show that enhanced delivery to brain can be achieved following nasal administration of phospholipids/active oil compositions with fluorescein isothiocyanate (FITC) and near—infrared (NIR) probes.

The nasal delivery of the composition of the invention is especially suitable for CNS administration for treating diseases. The invention therefore also provides a method of treating one or more conditions and diseases in a subject, for example, degenerative diseases, Parkinson, anxiety, or reducing the risk to suffer from such diseases, or otherwise improving brain health, and in particular improving cognitive function and memory and decreasing cognitive function and memory decline (both due to normal aging and neurodegenerative diseases) comprising nasally administering to said subject a composition comprising one or more vegetable oils that possess medicinal properties, or isolated active fractions of such oils, in admixture with phospholipids and optionally glycol, as described above.

The following diseases and conditions can be mentioned in connection with the brain targeted therapy offered by the invention: neurological disorder, memory and cognitive decline due to disease or normal aging processes, insomnia, autism, pain, anxiety, migraine, dementia, Alzheimer, Parkinson, improving awakens, mood disorders, post-trauma.

### IN THE DRAWINGS

FIG. 1: Representative multiphoton micrographs for the olfactory region in mice brains treated nasally with 10 μl of Composition A or Control Composition, each containing 0.5% w/w FITC. Field of images: height: 818 μm, width: 818 μm for the two images and depth: 100 μm for Composition III and 200 μm for Control Composition; lens ×20 (A1-MP microscope NIKON-Japan).

FIG. 2: Representative NIR images for mice brains treated nasally with 10 μl of Composition B or Control Composition each containing 0.5% w/w ICG as compared to untreated mice.

FIG. 3: Mean time spent on the rotarod (Fall Latency on rotarod) for reserpinized mice: [symbol] treated with PSO nasal composition (n=6) and [symbol] untreated animals (n=5), (Mean±SD). p<0.05 for PSO nasal composition vs. untreated control by two-tailed Mann-Whitney test.

FIG. 4: Number of squares crossed in the open field test for reserpinized mice: [symbol] treated with PSO nasal composition (n=6) and [symbol] untreated animals (n=5), (Mean±SD). p<0.01 for PSO nasal composition vs. control by two-tailed Mann-Whitney test.

FIG. 5: Number of squares crossed in the open field test for reserpinized mice treated with: [symbol] PSO nasal composition (n=6); [symbol] PSO by oral administration (n=6) and [symbol] untreated animals (n=7), (Mean±SD). p<0.01 for PSO nasal composition vs. untreated control, p<0.05 for PSO nasal composition vs. PSO oral, for PSO oral vs. untreated control p>0.05 (considered not significant), by one-way ANOVA.

FIG. 6: Number of squares crossed in the open field test by reserpinized mice treated with: [symbol] PSO new nasal composition; [symbol] PSO by oral administration and [symbol] untreated animals (n=6/group) (Mean±SD). p<0.001 for PSO nasal composition vs. untreated control, p<0.05 for PSO nasal composition vs. PSO oral, p>0.05(considered not significant), for PSO oral vs. untreated control, by one-way ANOVA.

### EXAMPLES

Phospholipon 90 G is from Lipoid GmbH, Germany. Sunlipon® 50, Sunlipon® 65, Sunlipon® 90 are from Perimondo LLC, USA. Pomegranate Oil (organic) manufactured by Bara Herbs, Israel and Hemp Seed Oil (organic) manufactured by Pukka Herbs, UK, were used. Lecithin Soya is from Fagron, Spain and propylene glycol from Tamar, Israel. Olive Oil from Henry Lamotte Oil GmbH, Germany.

### Examples 1-9 Oil(s)—Containing Compositions

The compositions tabulated in Table 1 below were prepared by the methods described below.

| | 1 | 2 | 3 | 4 | 5 | 6 | 7 | 8 | 9 |
|---|---|---|---|---|---|---|---|---|---|
| Ingredient | wt % | | | | | | | | |
| Lecithin soya | 33.3 | 30.3 | 44.1 | 40 | 40 | 50 | | | |
| Phospholipon ® | | | | | | | 63.3 | 60.5 | 60.0 |
| 90G | | | | | | | | | |
| Pomegranate oil | 66.7 | | 33.9 | 20 | 20 | 25 | 5.5 | 9.5 | |
| Black sesame | | 69.7 | | | | | | | |
| seed oil | | | | | | | | | |
| Olive oil | | | 22.0 | 40 | | | | | |
| Black cumin | | | | | | | | | 8.8 |
| seed oil | | | | | | | | | |
| Hemp seed oil | | | | | 40 | 25 | | | |
| Propylene | | | | | | | 31.2 | 30.0 | 31.2 |
| glycol | | | | | | | | | |

The compositions of Examples 1 to 6 were prepared by mixing lecithin with a portion of the oil followed by gradual addition of the remaining amount of the oil under stirring (and the second oil, if a pair of oils is used). Homogeneous brownish viscous liquids were obtained.

The compositions of Examples 7 to 9 were prepared by mixing phospholipon 90G with propylene glycol to obtain a homogeneous gel, followed by gradual addition of the oil under stirring. Homogeneous viscous yellowish or brownish liquids were formed.

### Examples 10-14 Oil-Containing Compositions

TABLE 2

| | 10 | 11 | 12 | 13 | 14 |
|---|---|---|---|---|---|
| Ingredient | wt % | | | | |
| Phospholipon ® 90G | | | | 35 | 30 |
| Sunlipon ® 50 | 33 | | | | |
| Sunlipon ® 65 | | 28 | | | |
| Sunlipon ® 90 | | | 33 | | |
| Pomegranate oil | 67 | 72 | 67 | 65 | 70 |

The compositions of Examples 10 to 14 were prepared by mixing phospholipid with a portion of the oil using mortar and pestle followed by gradual addition of the remaining amount of the oil under mixing. Homogeneous yellowish to brownish viscous liquids were obtained.

### Examples 15 (of the Invention) and 16 (Comparative) Nasal Delivery to the Brain of Oils-Containing Compositions Measured by Multiphoton Imaging

The compositions tabulated in the Table below were prepared. These compositions were nasally administered to mice; their delivery to the brain via the nasal route was measured by multiphoton imaging.

TABLE 3

| | Example 15 | Example 16 |
|---|---|---|
| | Composition A | (control) |
| Ingredient | Wt % | |
| Lecithin soya | 40.0 | |
| Pomegranate oil | 20.0 | 20.0 |
| Sesame oil | 39.0 | 49.0 |
| α-tocopherol | 0.5 | 0.5 |
| FITC | 0.5 | 0.5 |
| Vaseline | | 30.0 |

### Preparation

Composition A (of Example 15) was prepared in the following way. Lecithin was mixed with pomegranate oil, followed by addition of α-Tocopherol. Then sesame oil was added gradually under mixing. Lastly, FITC was added under mixing.

The Control Composition (of Example 16) was prepared in the following way. Vaseline was mixed with pomegranate oil, followed by addition of α-Tocopherol. Sesame oil was then added slowly under mixing. Lastly, FITC was added and mixed well.

### Experimental Protocol

Mice were treated with 10 μl of Nasal Composition A or Control Composition each containing 0.5% w/w FITC. Ten minutes after treatment, the animals were sacrificed; brains were removed, washed with normal saline and the olfactory region in brain was observed under the multiphoton microscope A1-MP microscope (NIKON, Japan). The field of image was 818×818×200 nm (width×height×depth), the scanning was performed using objective lens ×20, excitation wavelength of 740 nm, laser intensity 6%, scan speed 0.125. The fluorescence intensity of the probe (Arbitrary units, A.U.) in scanned brain region was further analyzed using ImageJ software. The brain of untreated mouse was examined to rule out the auto-fluorescence of the olfactory region.

### Results

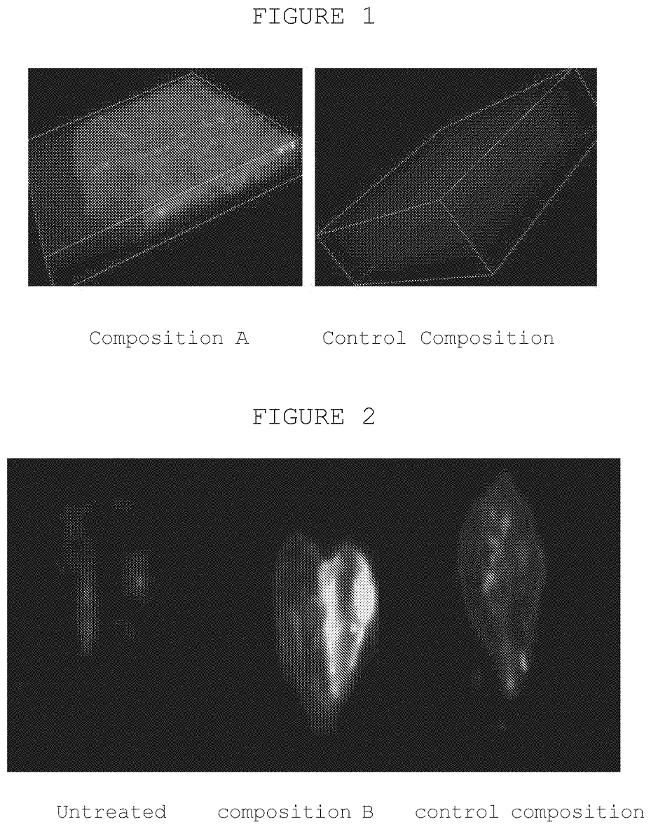

The multiphoton micrographs obtained in this experiment are given in FIG. 1. The results show that the nasal administration of Composition A (containing FITC) yielded a strong fluorescent signal as compared to Control FITC Composition. Semi-quantification of the images shows a fluorescent intensity of 51.7 A.U. for Composition A. On the other hand, nasal administration of FITC from the Control Composition yielded a fluorescent intensity of only 9.4 A.U.

### Examples 17 (of the Invention) and 18 (Comparative) Nasal Delivery to the Brain of Oils-Containing Compositions Measured by NIR Imaging

The compositions tabulated in Table 4 below were prepared. These compositions were nasally administered to mice. The delivery of the NIR probe Indocyanine green (ICG) to the cortex in mice brain from nasal compositions of the invention was examined by Odyssey® Infrared Imaging System (LI-COR, USA).

TABLE 4

| | Example 17 | Example 18 |
|---|---|---|
| | Composition B | (Control) |
| | % w/w | % w/w |
| Phospholipon 90G | 19.0 | |
| Pomegranate oil | 5.0 | |
| Propylene glycol | 75.0 | 99.5 |
| α-tocopherol | 0.5 | |
| ICG | 0.5 | 0.5 |

Composition B (of Example 17) was prepared in the following way. Phospholipon was dissolved in propylene glycol followed by addition of α-Tocopherol. Then pomegranate oil was added under mixing. Lastly, ICG was added and mixed well.

The Control Composition (of Example 18) was prepared by dissolving the ICG in propylene glycol.

### Experimental Protocol

Mice were treated with 10 μl of Nasal Composition B or Control Composition, each containing 0.5% w/w ICG as compared to untreated mice. Thirty minutes after treatments, the animals were sacrificed; brains were removed, washed with normal saline and observed under the imaging system. The scanning was performed using offset 2, resolution 339.6 μm, channel 800 nm and intensity 1. The fluorescence intensity of the probe (Arbitrary units, A.U.) in brain was further analyzed using ImageJ software.

### Results

The NIR images obtained in this experiment show that the nasal administration of Composition B yielded a strong fluorescent signal as compared to Control ICG Composition (FIG. 2). Semi-quantification of the images and normalization of the fluorescence intensity (by subtracting the auto fluorescence of the untreated brain from the fluorescence intensity of each image) show a fluorescent intensity of 7.5 A.U. for Composition B. On the other hand, nasal administration of the Control ICG Composition yielded a fluorescent intensity of only 0.4 A.U.

### Example 19

### In Vivo Testing of the Effect of Nasal Administration of pomegranate seed oil (PSO) composition of the invention on the anxiety and motor behavior of reserpinized mice

The goal of the study reported herein was to evaluate the effect on the anxiety and motor behavior of reserpinized mice of PSO composition of the invention administrated nasally.

Materials Materials used in this study are set out in Table 5.

TABLE 5

| Material | Abbreviation | Manufacturer |
|---|---|---|
| Pomegranate seed oil | PSO | N.S. Oils Ltd¹ |
| Lecithin soya | Lecithin | Fagron² |
| Reserpine | — | Sigma Aldrich Israel¹ |
| Dimethyl sulfoxide | DMSO | Bio Lab Ltd¹ |
| Tween 80 | — | Sigma Aldrich Israel¹ |

¹Israel
²Spain

The PSO composition is tabulated in Table 6.

TABLE 6

| Ingredient | Concentration % w/w |
|---|---|
| Lecithin | 33 |
| PSO | 67 |

### Experimental Protocol

### Animals

All procedures carried out on animals were according to The National Institutes of Health regulations and were approved by the Committee for Animal Care and Experimental Use of the Hebrew University of Jerusalem.

The experiment was performed on 11 male CD-1 ICR mice (28-33 g). Mice were housed under standard conditions of light and temperature in plastic cages in the specific-pathogen unit (SPF) of the pharmacy school at the Hebrew University of Jerusalem. Animals were kept in separated cages with smooth flat floor and provided with unlimited access to water and food.

### Treatments

The mice were divided randomly into one group treated nasally with PSO composition of Table 6 (n=6) and untreated control group (n=5). Animals in the treatment group received PSO from the composition of the invention at a dose of 300 mg/kg twice daily for 5 days then last dose was given on the sixth day one hour before the behavioral testing.

On the fifth day of the experiment the animals of the two groups received intraperitoneal injection of Reserpine at a dose of 3 mg/kg from a 0.03% Reserpine solution. Reserpine injection was prepared by suspending Reserpine in DDW containing 0.1% DMSO and 0.3% Tween 80.

### Rotarod Test

To assess the motor coordination of the animals, an accelerating Rotarod device (Rotarod for mouse, Model 47650, UGO Basile S.R.L. Italy) was used. The test was carried out 21 hours after Reserpine injection and 1 hour after the last administration of PSO nasal composition. The Rotarod test consisted of a suspended rod on which each individual mouse was placed. The rod was accelerated during 180 sec from 5 rounds per minute (RPM) to 55 RPM. The trial was stopped when the mouse fell off the Rotarod or after completing the 180 sec (cut off). The mean of three trials was taken. One hour prior to the Reserpine injections, the mice were trained to perform the test.

### Open Field Test

Spontaneous locomotor activity was measured 22 hours after reserpine injection and 2 hours after last administration of PSO nasal composition. Mice were placed in the center of a cage (29×28.5×30 cm) with the floor divided into nine equal squares. The number of squares crossed by the animal was counted during 5 min with no habituation session.

### Results

The results of the in vivo experiment measuring the effect of nasal administration of PSO composition of invention are presented in Tables 7 and 8 and Figures below.

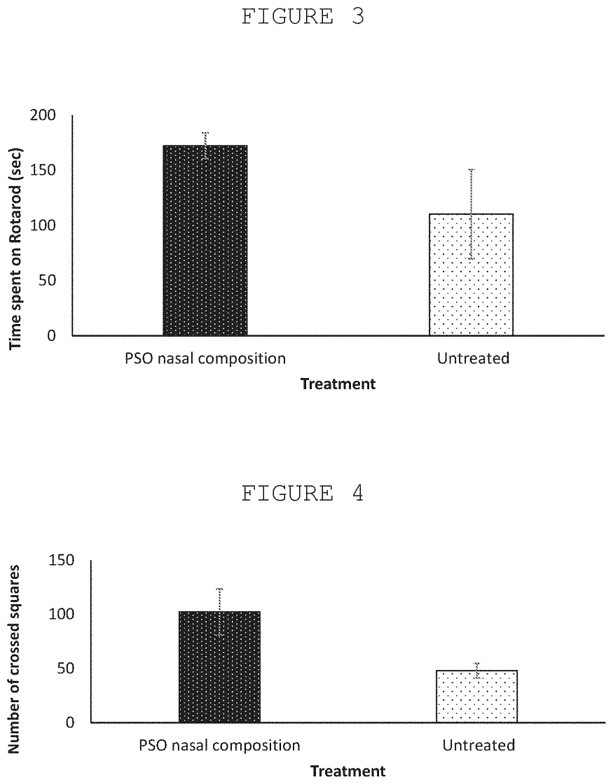

Table 7 shows the mean time spent on the rotarod (Fall Latency on rotarod) for reserpinized mice treated nasally with PSO nasal composition (n=6) and untreated animals (n=5), (Mean±SD). The results are also shown in the bar diagram of FIG. 3 (left and right bars for the treated and non-treated groups, respectively).

TABLE 7

| | Group | |
|---|---|---|
| | PSO nasal | |
| | composition | Untreated |
| Time spent on | 172.3 ± 11.7 | 110.3 ± 40.4 |
| Rotarod (sec) | | |

Table 8 shows the number of squares crossed in the open field test by reserpinized mice treated with PSO nasal composition (n=6) and untreated animals (n=5), (Mean±SD). The results are also shown in the bar diagram of FIG. 4 (left and right bars for the treated and non-treated groups, respectively).

TABLE 8

| | Group | |
|---|---|---|
| | PSO nasal | |
| | composition | Untreated |
| Number of | 102.2 ± 21.3 | 48.0 ± 6.9 |
| squares crossed | | |

These data suggest that nasally administrated PSO composition is able to reverse the anxiety and increase the locomotor activity in animal model.

The data obtained from the Rotarod test show that nasal administration of PSO composition for six days lead to significant increase in the locomotor activity of reserpinized mice which was expressed by longer time spent on the rotarod device. Animals treated with PSO nasal composition spent 172.3±11.7 sec on the rotarod compared to untreated reserpinized animals which spent only 110.3±40.4 sec (p<0.05).

These results are confirmed by the data obtained in the open field test where higher locomotor activity expressed by a very significant (p<0.01) higher number of crossed squares, 102.2±21.3 was observed in the mice treated nasally with the PSO formulation of the invention. The untreated reserpinized animals crossed only 48.0±6.9 squares.

### Example 20

### In Vivo Testing the Effect of Nasal Administration of PSO Composition of the Invention Versus PSO Oral Administration on the Anxiety and Motor Behavior

The goal of the study reported herein was to evaluate the effect of PSO nasal composition on the anxiety and motor behavior of reserpinized mice in comparison with oral administration of PSO. In this experiment the effect of PSO was tested 2.5 hour after the administration of the last dose oil formulation in reserpinized mice with 3 mg/kg Reserpine.

### Materials

Materials used in this study are set out in Table 5 Example 19.

### Experimental Protocol

### Animals

All procedures carried out on animals were according to The National Institutes of Health regulations and were approved by the Committee for Animal Care and Experimental Use of the Hebrew University of Jerusalem.

The experiment was performed on 19 male CD-1 ICR mice (24-29 g). Mice were housed under standard conditions of light and temperature in plastic cages in the specific-pathogen unit (SPF) of the pharmacy school at the Hebrew University of Jerusalem. Animals were kept in separated cages with smooth flat floor and provided with unlimited access to water and food.

### Treatments

The mice were divided randomly into two PSO treated groups, PSO nasal composition of the invention (n=6), PSO oral (n=6) and one untreated control group (n=7). Animals in the treatment groups received PSO nasally from the composition of the invention or PSO orally at a dose of 300 mg/kg twice daily for 5 days, then the last dose was given on the sixth day 2.5 hours before the behavioral test.

On the fifth day of the experiment, the animals of the three groups received an intraperitoneal injection of Reserpine 3 mg/kg. Reserpine injection was prepared in DDW containing 0.1% DMSO and 0.3% Tween 80.

### Open Field Test

Spontaneous locomotor activity of animals was measured 23 hours after reserpine injection and 2.5 hours after last administration of PSO. Mice were placed in the center of a cage (29×28.5×30 cm), with the floor divided into nine equal squares. The number of squares crossed was counted during 5 min with no habituation session.

### Results

The results of this experiment are presented in Table 9 and FIG. 5.

Table 9 shows the number of squares crossed in the open field test for reserpinized mice treated with PSO new nasal composition (n=6), PSO by oral administration (n=6) and untreated animals (n=7), (Mean±SD). The results are also shown in the form of a bar diagram in FIG. 5 (left, middle and right bars stand for PSO nasal composition, PSO oral treatment and the non-treated groups, respectively).

TABLE 9

| | Group | | |
|---|---|---|---|
| | PSO nasal | | |
| | composition | PSO oral | Untreated |
| Number of | 171.3 ± 33.6 | 106.2 ± 42.5 | 90.0 ± 30.2 |
| squares crossed | | | |

It is seen that mice treated with PSO nasal composition expressed higher locomotor activity and crossed 171.3±33.6 squares. The animals in the PSO oral group crossed only 106.2±42.5 squares and the untreated animals 90.0±30.2 squares. The results are significant: p<0.05 for PSO nasal composition versus PSO oral and p<0.01 for PSO nasal composition versus untreated control. These results indicate the superiority of the effect obtained with the nasal administration of PSO composition in comparison with oral administration of PSO.

### Example 21

### Effect on the Anxiety and Motor Behavior of Reserpinized Mice of Nasal Administration of PSO Nasal Composition Comparative to Oral Administration of PSO

The goal of the study was to evaluate the effect of PSO nasal composition on the anxiety and motor behavior of reserpinized mice in comparison with oral administration of PSO.

In this experiment the effect of PSO was tested 1.5 hour after the administration of the last dose in mice that received 4 mg/kg Reserpine.

### Materials

Materials used in this study are set out in Table 5 Example 19.

### Experimental Protocol

### Animals

All procedures carried out on animals were according to The National Institutes of Health regulations and were approved by the Committee for Animal Care and Experimental Use of the Hebrew University of Jerusalem.

This experiment was performed on 18 male CD-1 ICR mice (25-31 g). Mice were housed under standard conditions of light and temperature in plastic cages in the specific-pathogen unit (SPF) of the pharmacy school at the Hebrew University of Jerusalem. Animals were kept in separated cages with smooth flat floor and provided with unlimited access to water and food.

### Treatments

The mice were divided randomly into two PSO treated groups, PSO nasal composition of the invention, PSO oral and one untreated control group, (n=6/group). Animals in the treatment groups received PSO from the composition of the invention or PSO orally at a dose of 300 mg/kg twice daily for 5 days, then last dose was given on the sixth day 1.5 hours before the test.

On the fifth day of the experiment, the animals of the three groups received intraperitoneal injection of 4 mg/kg Reserpine. The injection was prepared by suspending Reserpine in DDW containing 0.1% DMSO and 0.3% Tween 80.

### Open Field Test

The spontaneous locomotor activity of the animals was measured 23 hours after reserpine injection and 1.5 hours after last administration of PSO. Mice were placed in the center of a cage (29×28.5×30 cm), with the floor divided into nine equal squares. The number of squares crossed by the animal was counted during 5 min with no habituation session.

### Results

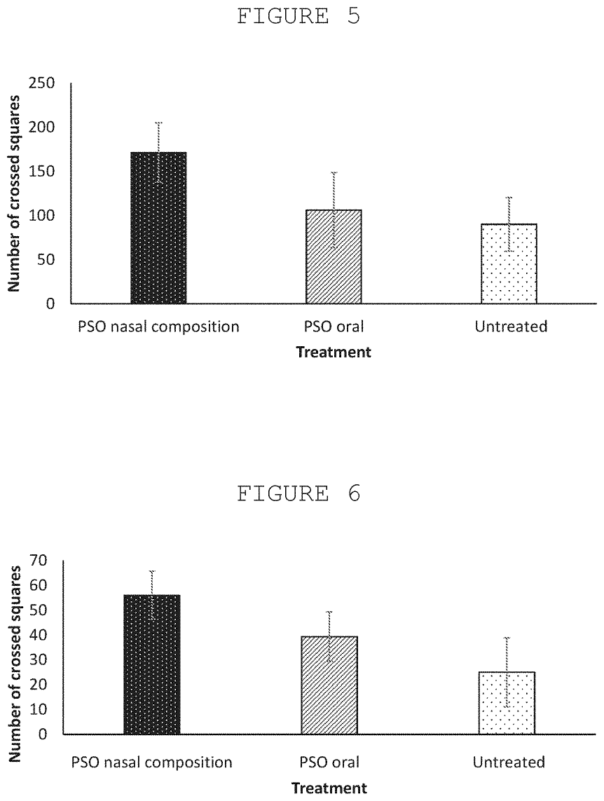

The results of this in vivo experiment are presented in Table 10 and FIG. 6.

Table 10 shows the number of squares crossed in the open field test for reserpinized mice treated with PSO new nasal composition, with PSO by oral administration and untreated animals (n=6/group), (Mean±SD). The results are also shown in the form of a bar diagram in FIG. 6 (left, middle and right bars stand for PSO nasal composition, PSO oral treatment and the non-treated groups, respectively).

TABLE 10

| | Group | | |
|---|---|---|---|
| | PSO nasal | | |
| | composition | PSO oral | Untreated |
| Number of | 56.6 ± 9.7 | 39.3 ± 10.0 | 25.0 ± 13.9 |
| squares crossed | | | |

These results confirm the data obtained in Example 20 where the PSO nasal composition increased the number of crossed squares by ˜200%. These finding emphasizes the effect of the nasal PSO composition to reverse the effects of reserpine and enhance the locomotor activity in the tested animal model.

Mice treated with PSO new nasal composition expressed increase locomotor activity by crossing 56.6±9.7 squares. The PSO oral group only 39.3±10.0 and untreated animals 25.0±13.9 squares. The results are significant: p<0.05 for PSO nasal composition versus PSO oral and p<0.001 for PSO nasal composition versus untreated control.

1. A nasal composition comprising one or more vegetable oils that possess medicinal properties for the treatment or improvement of brain/central nervous system conditions and diseases; phospholipids and optionally glycol, the composition being cannabinoids-free.

2. A composition according to claim 1, wherein the oil is selected from the group consisting of pomegranate seed oil, black cumin seed oil, hemp seed oil, sesame seed oil, black sesame oil and mixtures thereof.

3. A composition according to claim 2, comprising pomegranate oil.

4. The composition of claim 3, wherein the concentration of the pomegranate seed oil is from 1% to 75% based on the total weight of the composition.

5. The composition according to claim 1, wherein the concentration of the phospholipids is from 15 to 70% by weight.

6. The composition of claim 4, comprising:
from 40 to 70% by weight of pomegranate seed oil or a mixture thereof with a second oil; and
from 30 to 60% of phospholipids.

7. The composition of claim 6, comprising:
from 50 to 70% by weight of pomegranate seed oil; and
from 30 to 40% of phospholipids.

8. The composition of claim 4, comprising:
from 1 to 20% by weight of pomegranate seed oil;
from 15 to 70% of phospholipids; and
from 20 to 75% of propylene glycol.

9. A method of treating one or more conditions and diseases in a subject, selected from degenerative diseases and anxiety, or reducing the risk to suffer from such diseases and conditions, the method comprises nasally administering to said subject a composition according to claim 1.

10. A method of improving cognitive function and memory, or reducing cognitive function and memory decline in a subject, comprising nasally administering to said subject a composition according claim 1.
